(19) United States
(12) Patent Application Publication
(10) Pub. No.: US 2023/0033795 A1
(43) Pub. Date: Feb. 2, 2023

(54) NEW THERAPY

(71) Applicant: UCL Business Ltd, London (GB)

(72) Inventor: Veronica KINSLER, London (GB)

(21) Appl. No.: 17/786,187

(22) PCT Filed: Dec. 15, 2020
(86) PCT No.: PCT/GB2020/053216

§ 371 Date: Jun. 16, 2022

(30) Foreign Application Priority Data
Dec. 18, 2019 (GB) 1918752.5

(51) Int. Cl. A61K 31/575; A61P 17/06; A61K 31/366

(52) U.S. Cl. CPC A61K 31/575; A61P 17/06; A61K 31/366

(57) ABSTRACT

The present invention provides a pharmaceutical composition which comprises: (a) an HMG-CoA reductase inhibitor; and (b) cholesterol or a pharmaceutically acceptable precursor thereof; for use in treating psoriasis.

The present invention relates to the treatment of psoriasis. In particular, the treatment involves the use of a combination of an HMG-CoA reductase inhibitor and cholesterol or a precursor thereof.

### BACKGROUND TO THE INVENTION

Psoriasis is a common skin disease of unknown etiology which can affect children as well as adults. It consists of inflamed, red thickened, scaly skin, usually but not always in a plaque type distribution. When severe or extensive it can be difficult to treat, often requiring systemic therapies with their attendant risks of side effects.

New treatments for this condition would be highly desirable. Particularly desirable would be treatment strategies based on the use of drugs already validated for treatment of other conditions and/or substances known to be pharmacologically acceptable owing to their endogenous existence. In addition, particularly desirable would be new topical therapies, as in general these are considered to be less of an intervention, easily administered, and with fewer side effects than systemic therapies, and do not require healthcare worker administration.

It has now been found that a combination of an HMG-CoA reductase inhibitor and cholesterol, or a precursor thereof, may have significant efficacy in treating psoriasis. The activity of the combination therapy may in particular be higher than would be expected from the activity of either component of the combination, when used as a monotherapy. Topical administration of the combinations defined above may also avoid side effects or metabolic processing before reaching the skin resulting from systemic administration, as well as improving efficacy at a given dosage.

The combination therapy may, in particular, be beneficial for psoriasis patients having a particular genetic profile, and specifically having one or more variants in genes associated with cholesterol metabolism. Thus, it has now been found that the manifestation of psoriasis, at least in a proportion of cases, may be associated with genetic or otherwise induced imbalances in cholesterol metabolism, or genetic or otherwise induced defects leading to functional effects on the cholesterol metabolism process.

### SUMMARY OF THE INVENTION

The present invention provides a pharmaceutical composition which comprises: (a) an HMG-CoA reductase inhibitor; and (b) cholesterol or a pharmaceutically acceptable precursor thereof; for use in treating psoriasis.

The present invention also provides an HMG-CoA reductase inhibitor for use in treating psoriasis, by co-administration with cholesterol or a pharmaceutically acceptable precursor thereof.

The present invention additionally provides cholesterol or a pharmaceutically acceptable precursor thereof for use in treating psoriasis, by co-administration with an HMG-CoA reductase inhibitor.

The present invention still further provides a method of treating a patient suffering from psoriasis which method comprises co-administering to said patient (a) an HMG-CoA reductase inhibitor, and (b) cholesterol or a pharmaceutically acceptable precursor thereof.

Also provided by the present invention is a product comprising (a) an HMG-CoA reductase inhibitor, and (b) cholesterol or a pharmaceutically acceptable precursor thereof, as a combined preparation for simultaneous, concurrent, separate or sequential use in the treatment of a patient suffering from or susceptible to psoriasis.

Furthermore, the present invention provides the use of (a) an HMG-CoA reductase inhibitor in the manufacture of a medicament for the treatment of psoriasis by co-administration with (b) cholesterol or a pharmaceutically acceptable precursor thereof.

The present invention additionally provides use of (b) cholesterol or a pharmaceutically acceptable precursor thereof in the manufacture of a medicament for the treatment of psoriasis by co-administration with (a) an HMG-CoA reductase inhibitor.

### BRIEF DESCRIPTION OF THE FIGURES

FIG. 1 shows the results of Filipin III staining of cultured and immortalized primary keratinocytes from psoriasis patients (left side) and control patients (right side), as described in more detail in Example 1 below.

FIG. 2 shows the results of a WST-1 proliferation assay from psoriasis patients, both untreated (left side; control) and treated with a combination of cholesterol and simvastatin (right side), as described in more detail in Example 3 below.

FIG. 3 shows total cholesterol (panel A) and vitamin D3 (panel B) concentrations of keratinocytes isolated from three psoriasis (right side) and ten control patients (left side) measured respectively using Gas Chromatography Mass Spectrometry (GC-MS) and Supercritical Fluid Chromatography Mass Spectrometry (SFC-MS), normalised to live cell count, and as described in Example 3 below.

### DETAILED DESCRIPTION OF THE INVENTION

HMG-CoA Reductase Inhibitor

An HMG-CoA reductase inhibitor is a substance that is capable of inhibiting HMG-CoA reductase. HMG-CoA reductase is the rate-controlling enzyme of the mevalonate pathway, which produces cholesterol and other isoprenoids in vivo. HMG-CoA reductase inhibitors are commonly known as statins, and the terms HMG-CoA reductase inhibitor and statin are used interchangeably in the present disclosure.

As will be well known by those skilled in the art, statins have been widely prescribed for reducing serum cholesterol levels in patients in need thereof. For example, they have been utilised to reduce the risk of heart disease in individuals with high cholesterol, to reduce mortality in patients having existing cardiovascular disease and in various other conditions associated with undesirably high cholesterol levels.

Non-limiting examples of HMG-CoA reductase inhibitors that can be used in accordance with the present invention include atorvastatin, cerivastatin, fluvastatin, lovastatin, mevastatin, pitavastatin, pravastatin, rosuvastatin, and simvastatin, as well as pharmaceutically acceptable salts and esters thereof. Presently preferred HMG-CoA reductase inhibitors include atorvastatin, fluvastatin, pravastatin, rosuvastatin, simvastatin and lovastatin, and pharmaceutically acceptable salts and esters thereof. Particularly preferred is simvastatin and lovastatin, or a pharmaceutically acceptable salt or ester thereof. For example, the HMG-CoA reductase inhibitor may be simvastatin or lovastatin.

It is within the scope of the present invention to make use of two or more such compounds. Thus, the HMG-CoA reductase inhibitor may comprise a single active agent (i.e. a single statin) or it may comprise two or more active agents (i.e. two or more statins).

Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002. Suitable pharmaceutically acceptable salts of the compounds for use in this invention include addition salts with a pharmaceutically acceptable acid such as such as hydrochloric acid, sulphuric acid, methanesulphonic acid, fumaric acid, maleic acid, succinic acid, acetic acid, benzoic acid, citric acid, tartaric acid or phosphoric acid. Other salts may be formed with a pharmaceutically acceptable base. Suitable such pharmaceutically acceptable salts include alkali metal salts, e.g. sodium or potassium salts; alkaline earth metal salts, e.g. calcium or magnesium salts; ammonium salts; and salts formed with suitable organic ligands, e.g. quaternary ammonium salts, and meglumine salts.

Standard principles similarly underlie the selection and preparation of pharmaceutically acceptable esters. If a HMG-CoA reductase inhibitor contains more than one hydroxyl moiety, then one or more than one (for example all) of the hydroxyl moieties may be esterified. Examples of suitable esters include C₁₋₆ alkynyl, alkenyl and alkyl esters or such esters in which one of the carbon atoms of the C₁₋₆ alkynyl, alkenyl or alkyl group (along with any hydrogen atoms to which it is attached) is replaced by phenyl. Specific examples include C₁₋₆ alkyl and phenyl esters, e.g. C₁₋₄ alkyl esters (such as methyl, ethyl, propyl, isopropyl, butyl, isobutyl or tert-butyl ester) and phenyl esters.

Cholesterol or Precursor Thereof

Cholesterol is one of the three key stratum corneum lipids (along with ceramides and free fatty acids) that form the extracellular lamellar bilayer that mediates epidermal barrier function.

Cholesterol is an endogenous sterol that has the chemical formula

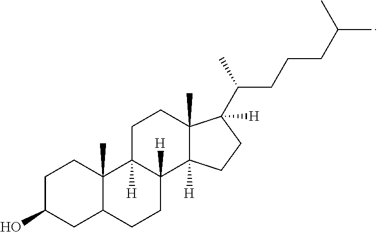

In accordance with the present invention the cholesterol is most preferably provided as such as an active ingredient. However, a pharmaceutically acceptable precursor of cholesterol can also be used, provided that such a precursor is capable of generating cholesterol in vivo in the subject after administration of the precursor. It is also possible to use a plurality of such compounds, e.g. cholesterol in combination with one or more pharmaceutically acceptable precursors of cholesterol, or two or more pharmaceutically acceptable precursors of cholesterol.

A pharmaceutically acceptable precursor of cholesterol is a pharmaceutically acceptable substance that is capable of metabolising in order to form cholesterol after administration to a patient (i.e., in vivo). Non-limiting examples of such precursors include a prodrug of cholesterol and an intermediate in the in vivo production of cholesterol from mevalonate.

A prodrug is a (typically synthetic) derivative of cholesterol that is capable of metabolising to form cholesterol after administration. Often the prodrug of cholesterol is a compound in which the hydroxyl moiety of cholesterol is derivatised, such as esterified. Non-limiting examples of suitable prodrugs thus include a cholesterol ester, a cholesterol phosphate ester and a cholesterol sulphate ester.

The prodrug, may, for example, be a compound of the formula (I)

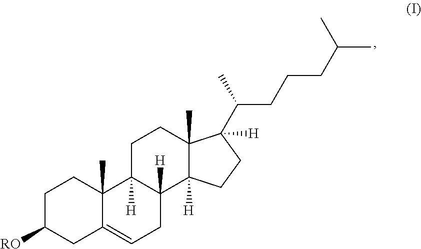

in which the group —OR is an ester group, a phosphate ester group or a sulphate ester group. One preferred class of prodrug is a cholesterol ester of formula (I), in which R is a hydrocarbyl group, for example a C₁₋₂₀ alkynyl, alkenyl or alkyl group or such a group in which one to three of the carbon atoms (along with any hydrogen atoms to which it is attached) is replaced by C₆₋₂₀ aryl. Examples include C₁₋₆ alkynyl, alkenyl and alkyl or such a group in which one of the carbon atoms (along with any hydrogen atoms to which it is attached) is replaced by phenyl. Specific examples include C₁₋₆ alkyl and phenyl, e.g. C₁₋₄ alkyl (such as methyl, ethyl, propyl, isopropyl, butyl, isobutyl or tert-butyl ester) and phenyl.

An intermediate in the in vivo production of cholesterol from mevalonate is an endogenous cholesterol precursor compound. The biosynthetic pathway by which cholesterol is produced in vivo from mevalonate is well understood and proceeds via a well-known cascade of chemical reactions and intermediate compounds. Any intermediate compound in this well-known biosynthetic pathway can be utilised as the intermediate in accordance with the present invention.

Examples of such intermediates that can be used in accordance with the invention include mevalonate itself (and mevalonic acid), mevalonate-5-phosphate, mevalonate pyrophosphate, isopentyl pyrophosphate (IPP), dimethylallyl pyrophosphate (DMAPP), geranyl pyrophosphate, farnesyl pyrophosphate, squalene, 2,3-oxidosqualene, lanosterol, 4,4-dimethylcholesta-8(9),14,24-trien-3β-ol, 4,4-dimethylcholesta-8(9),24-dien-3β-ol, zymosterol, cholesta-7,24-dien-3β-ol, cholesta-5,7,24-trien-3β-ol, desmosterol, 4,4,14α-trimethylcholesta-8(9)-en-3β-ol, 4,4-dimethylcholesta-8(9),14-dien-3β-ol, 4,4-dimethylcholesta-8(9)-en-3β-ol, cholesta-8(9)-en-3β-ol, lathosterol and 7-dehydrocholesterol (7DHC). A preferred such compound is 7-dehydrocholesterol. Pharmaceutically acceptable salts and esters of these compounds are also considered to be suitable intermediates within this disclosure. Examples of suitable salts and esters are the same as those disclosed elsewhere herein with reference to the HMG-CoA reductase inhibitor and the cholesterol prodrug.

Combination of Active Ingredients

The present invention involves the use of a combination of (a) an HMG-CoA reductase inhibitor, and (b) cholesterol or a pharmaceutically acceptable precursor thereof.

Such a combination of active ingredients has previously been reported to have a positive effect in a compassionate-use treatment of patients with CHILD syndrome (Kiritsi et al., Orphanet Journal of Rare Diseases 2014 9:33) and, more recently, in a small group of patients suffering from porokeratosis (Atzmony et al., J Am Acad Dermatol. 2019 Aug. 23. pii: S0190-9622(19)32648-9).

CHILD syndrome and porokeratosis are distinct conditions from psoriasis, differing both in terms of clinical diagnosis and general understanding amongst dermatologists. Additionally, it is notable that the authors of the Kiritsi et al. paper found that the specific composition found to be effective in therapy of CHILD patients was ineffective in the treatment of at least two other conditions closely related to CHILD (namely, X-linked recessive ichthyosis and Autosomal recessive congenital ichthyosis), thus emphasizing an inherent uncertainty in the applicability of the composition for treatment of other pathological conditions.

Without being limited to theory, it is considered that the efficacy of the present combination therapy in treating psoriasis may be linked to the patient group in question having abnormal cholesterol metabolism (i.e., as a consequence of single or multiple variants, new or described, rare or common, in one or more genes associated with cholesterol metabolism, such as those outlined in further detail elsewhere herein).

In one aspect, the present invention provides a pharmaceutical composition which comprises: (a) an HMG-CoA reductase inhibitor; and (b) cholesterol or a pharmaceutically acceptable precursor thereof; the pharmaceutical composition being for use in treating psoriasis. Pharmaceutical compositions according to the invention will typically further comprise one or more pharmaceutically acceptable excipients or carriers.

Typically the patient to be treated is a mammal. Preferably the patient is a human.

It will be appreciated that not all patients suffering from a particular condition (e.g. psoriasis) will necessarily experience (e.g., substantial) therapeutic benefits as a result of the present combination therapy. For the avoidance of doubt, it is not essential to the invention that every patient clinically diagnosed with psoriasis be susceptible to such treatment. Indeed, it is well established across the medical field as a whole that valuable therapeutic strategies will in general function more successfully in some patients than in others (and that in some patients, within the patient population as a whole, substantially no benefit may be observed). The combination therapy of the present invention provides a valuable alternative therapeutic strategy for the treatment of psoriasis.

Particularly preferred patients to be treated are those having functionally abnormal cholesterol metabolism in the skin. A patient having abnormal cholesterol metabolism may, for example, be defined as a patient having a variant in one or more genes associated with cholesterol metabolism. As used herein, a variant in a gene is an alteration in the most common DNA nucleotide sequence of the relevant gene. Variants can correspond to alternations that are benign, pathogenic or of unknown significance.

The variant or variants may, in general, be in any gene or genes associated with cholesterol metabolism or its control. Many such genes are already well known in the art. Without being limited by theory, the combination therapy may serve to correct cholesterol imbalances via the supply of exogenous cholesterol, as well as mitigating the deleterious effects arising from the genetic variant(s), including but not limited to the excessive formation of cholesterol precursors, by-products, or other related compounds, via the inhibition of HMG-CoA reductase and the resulting inhibition of the cholesterol pathway.

The variant in one or more genes associated with cholesterol metabolism may in some embodiments result in the gene in question having abnormally enhanced expression of its coded protein or abnormally suppressed expression of its coded protein (but at the most general level embraces any and all functionally significant effects on the one or more genes). The variant may be any change with respect to the relevant human genome reference sequence, particularly one having a functional consequence such as but not limited to abnormally enhanced expression of its coded protein or abnormally suppressed expression of its coded protein. For example, the variant may be (but is not limited to being) a single nucleotide variant (SNV), multiple nucleotide variant, a deletion variant, an insertion variant, a translocation, a missense variant or a splice site variant resulting in a change in the amino acid sequence (coding variant).

The one or more genes associated with cholesterol metabolism may be (but is/are not necessarily), for example, one or more genes selected from the genes listed in Table 1.

TABLE 1

AACS
AAGAB
ABCA1
ABCA12
ABCA13
ABCA2
ABCA5
ABCA7
ABCB4
ABCG1
ABCG4
ABCG5
ABCG8
ABHD5
ACAA2
ACADL
ACADVL
ACLY
ACSM1
ACSM3
ADAM17
ADIPOQ
AGMO
AGT
AGTR1
AKR1C1
AKR1D1
ALDH3A2
ALOX12B
ALOXE3
AMPD2
ANGPTL3
ANXA6
AP1S1
APOA1
APOA2
APOA4
APOA5
APOB
APOBR
APOC1
APOC2
APOC3
APOD
APOE
APOF
APOL1
APOL2
APOM
APP
AQP5
ARSE
ARV1
BCL10
C14orf1
CAD
CARD11
CARD14
CASP14
CAT
CAV1
CAV3
CBR3
CCL3
CCR5
CD24
CD36
CDSN
CEBPA
CEL
CELA3A
CELA3B
CERS3
CES1
CETP
CFTR
CH25H
CHUK
CLDN1
CLN6
CLN8
CLU
CNBP
CPS1
CSTA
CTSC
CUBN
CYB5R1
CYB5R2
CYB5R3
CYP11A1
CYP11B1
CYP11B2
CYP17A1
CYP19A1
CYP1B1
CYP21A2
CYP26B1
CYP27A1
CYP2C9
CYP39A1
CYP46A1
CYP4F22
CYP4V2
CYP51A1
CYP7A1
CYP7B1
CYP8B1
DGAT1
DGAT2
DHCR24
DHCR7
DHRS4
DISP3
DPYD
DSC2
DSC3
DSG1
DSG2
DSG4
DSP
DYNAP
EBP
EBPL
EGF
EHD1
EIF2A
ELOVL4
EPHX2
ERLIN1
ERLIN2
F7
FABP3
FABP4
FADS1
FAXDC2
FBXW7
FDFT1
FDPS
FDX1
FDX1L
FDXR
FGF1
FGFR4
G6PC
G6PD
GART
GGPS1
GJB2
GMPS
GPIHBP1
GPLD1
GPR183
HDLBP
HMGCR
HMGCS1
HMGCS2
HNF4A
HSD17B7
IDI1
IDI2
IKBKB
IKBKG
IL18
IL4
INHBA
INSIG1
INSIG2
JUP
KANK2
KRT1
KRT10
KRT2
KRT6C
KRT9
LAMTOR1
LBR
LCAT
LDLR
LDLRAP1
LEP
LEPR
LIPA
LIPC
LIPE
LIPG
LIPN
LMF1
LMNA
LOR
LPL
LRP1
LRP5
LRP6
LRP8
LSS
LYN
MALL
MALRD1
MALT1
MAP3K7
MBTPS1
MBTPS2
MED13
MIA2
MLC1
MSMO1
MSR1
MT3
MVD
MVK
MYLIP
NCEH1
NFKB1
NFKBIA
NIPAL4
NPC1
NPC1L1
NPC2
NR0B2
NR1D1
NR1H2
NR1H3
NR1H4
NR5A2
NSDHL
NUS1
OSBP
OSBP2
OSBPL10
OSBPL1A
OSBPL2
OSBPL3
OSBPL5
OSBPL7
OSBPL8
PCSK9
PDPK1
PEX7
PHYH
PKP1
PKP2
PLA2G10
PLA2G15
PLSCR3
PLTP
PMP2
PMVK
PNLIP
PNPLA1
POL32F
POMP
PON1
POR
PPARA
PPARD
PPARG
PRKAA1
PRKAA2
PRKAG2
PRKCQ
PROM2
PTCH1
RALY
RHBDF2
RIPK4
RORA
RORC
RXRA
SASH1
SC5D
SCAP
SCARB1
SCARF1
SCP2
SCP2D1
SEC14L2
SEC24A
SERPINA12
SHH
SIRT1
SLC27A4
SLURP1
SMAD2
SMO
SNAP29
SNX17
SOAT1
SOAT2
SOD1
SORL1
SPINK5
SQLE
SRD5A2
SREBF1
SREBF2
ST14
STAR
STARD3
STARD4
STARD5
STARD6
STS
STX12
SYP
SYT7
TGFB1
TGFBR1
TGFBR2
TGM1
TM7SF2
TMEM97
TNFSF4
TRAF6
TRERF1
TRPV3
TSPO
TSPO2
UMPS
URS00000E5433_9606
URS000013D17D_9606
URS0000272039_9606
USF1
USF2
VLDLR
VPS33B
VPS4A
VPS4B
XBP1
ZND750

All of the genes listed in Table 1 are known to be associated with cholesterol metabolism. In preferred aspects of the invention, the patients to be treated may have abnormal cholesterol metabolism. More preferably the patients to be treated may have a variant in one or more genes associated with cholesterol metabolism (e.g. a variant in one or more of the genes listed in Table 1). More preferably still the patients to be treated may have a variant in one or more genes selected from the group consisting of ABCG8, ACADL, APOF, CAD, CARD14, CBR3, CDSN, CLN8, CSTA, CYB5R2, CYP7B1, FBXW7, FDFT1, FDPS, FDX1L, HMGCR, KRT2, KRT6C, LDLR, LEP, LPL, LRP5, LRP8, OSBP, OSBP2, PKP1, PROM2, PTCH1, RORC, SCARB1, SERPINA12, SORL1, TRERF1, UMPS, TGFB1, PMVK, MVK and NSDHL. For instance, the patients to be treated may have a variant in one or more genes selected from the group consisting of ABCG8, ACADL, APOF, CAD, CARD14, CBR3, CDSN, CLN8, CSTA, CYB5R2, CYP7B1, FBXW7, FDFT1, FDPS, FDX1L, HMGCR, KRT2, KRT6C, LDLR, LEP, LPL, LRP5, LRP8, OSBP, OSBP2, PKP1, PROM2, PTCH1, RORC, SCARB1, SERPINA12, SORL1, TRERF1 and UMPS. Alternatively or additionally, the patients to be treated may have a variant in one or more genes selected from the group consisting of TGFB1, PMVK, MVK and NSDHL.

In a preferred embodiment, the patient may have no variant in the PMVK gene and/or the MVD gene.

In certain embodiments, the variant or variants may be “causative”. Causative variants are genetic variants occurring in genes that have a causative effect on a trait or condition in a subject (either as a single variant or in light of the combination of variants as a whole). A causative variant or variants in the context of the present invention thus means a genetic variant or variants that contributes to or results in the manifestation of psoriasis in the subject. The subject to be treated in the present invention may have such a causative variant or variants. The causative variant or variants is/are typically a mosaic variant or variants; it is, or they are, present in affected tissue, but not in unaffected tissue.

Causative variants for psoriasis may, for instance, be identified by taking biopsies and demonstrating that the variant is, or variants are, present in affected, but not unaffected, skin. The variants can be identified using methods known in the art such as by whole exome sequencing.

The subject to be treated may have at least one causative variant that contributes to or results in the manifestation of psoriasis in the subject. For the avoidance of doubt, references herein to the presence of “a” or “the” variant do not exclude the possibility that the subject exhibits a plurality of variants, e.g. two or more causative variants. The or each variant is typically a mosaic variant.

In some aspects of the disclosure, the subject does not have a CARD14 variant (e.g. a causative and/or mosaic CARD14 variant). In further aspects of the disclosure, the subject does not have a variant of one or more of PMVK, TGFB1, TGM1 and NSDHL (e.g. a causative and/or mosaic variant thereof).

The variant may, in certain aspects, be a gain-of-function variant or a loss-of-function variant. The variant is preferably a variant in a gene that modulates/alters cholesterol metabolism. Modulating/altering cholesterol metabolism may mean dysregulation therein, such as but not limited to insufficient cholesterol production, or excessive cholesterol production, or insufficient or excessive production of one or more metabolic cholesterol precursors.

Furthermore, homologs of the genes and proteins described herein may also be used in the present disclosure, i.e. they may also represent the gene or genes that are subject to a causative variant(s) in the subject. As used herein, “homology” refers to sequence similarity between a reference sequence and at least a fragment of a second sequence. As used herein, “homology” of a gene refers to the degree of identity of two or more gene sequences to each other. Thus, the higher the homology of two genes, the higher the identity or similarity of their sequences.

Whether two genes have homology can be examined by direct comparison of sequences, or by hybridization under stringent conditions in the case of nucleic acids. Homologs may be identified by any method known in the art, preferably, by using the BLAST tool to compare a reference sequence to a single second sequence or fragment of a sequence or to a database of sequences. As described below, BLAST will compare sequences based upon percent identity and similarity.

When directly comparing two gene sequences, the DNA sequence between the gene sequences is typically at least 50% identical, preferably at least 70% identical, more preferably at least 80%, 90%, 95%, 96%, 97%, 98% or 99% identical to each other, they have homology.

As used herein, “orthologue” refers to genes in different species that derive from a common ancestor gene. Homologous genes or homologous gene products are also sometimes referred to as orthologous genes or orthologous gene products. It is understood that such homologues, homologous gene products, orthologous genes or orthologous gene products and the like can also be used so long as they conform to the object of the present invention.

More generally, the manifestation of psoriasis in the patients to be treated may be associated with an imbalance/dysregulation of cholesterol metabolism, which may in turn have a number of contributory factors, including genetic factors (i.e. the presence of a genetic variant or combination of genetic variants as described herein) but optionally also one or more other factors such as environmental and behavioral factors (e.g., diet, lifestyle, etc.). These contributory factors may, in combination, lead to imbalances in cholesterol metabolism, and giving rise to a susceptibility to psoriasis or to psoriasis. Thus, the one or more genetic variants may play an at least contributory role in the manifestation of the psoriasis in the patients to be treated.

The psoriasis may, in general, be any clinical type of psoriasis. For instance, the psoriasis may be any form of psoriasis that is not pustular psoriasis. Examples of such psoriasis that may be treated include psoriasis vulgaris, guttate psoriasis, erythrodermic psoriasis, palmoplantar psoriasis, psoriatic arthritis (PsA) and inverse psoriasis. In some aspects of the disclosure, the psoriasis is not linear psoriasis; in some aspects the psoriasis is not ILVEN.

The present invention extends to situations where the active ingredients discussed above are co-administered. When the active ingredients are co-administered they can be present in separate pharmaceutical compositions. Thus, for example, the HMG-CoA reductase inhibitor can be administered orally, and the cholesterol or precursor thereof can be administered topically. Alternatively the HMG-CoA reductase inhibitor can be administered topically, and the cholesterol or precursor thereof can be administered orally. Still further, both components could be administered orally or, more preferably, topically in separate pharmaceutical compositions.

In a preferred embodiment, the active ingredients are formulated into a single pharmaceutical composition. Such a pharmaceutical composition may be suitable for administration by any appropriate means, including topically and orally. For some patients with very extensive disease, it may be more practical (and/or assist with patient compliance) to utilise oral administration. Preferably, however, the composition is suitable for topical administration. Topical administration of the HMG-CoA reductase inhibitor may be advantageous since it avoids the issue of first-pass hepatic metabolism of statins following systemic administration. Topical administration of the cholesterol or precursor thereof may also be advantageous since it avoids the issue of incorporation of systemically delivered compound into lipoprotein particles, which are unable to access peripheral tissues without LDL receptors, such as the epidermis.

For the avoidance of doubt, in the product comprising (a) an HMG-CoA reductase inhibitor, and (b) cholesterol or a pharmaceutically acceptable precursor thereof, as a combined preparation for simultaneous, concurrent, separate or sequential use in the treatment of a patient suffering from or susceptible to psoriasis, the product may comprise either a single pharmaceutical composition that comprises both (a) and (b) or alternatively a first pharmaceutical composition that comprises (a) and a second (i.e., separate) pharmaceutical composition that comprises (b).

Co-administration of the active ingredients according to the present invention includes simultaneous, separate and sequential administration. Typically, both drugs are administered simultaneously or one drug is administered first and the second drug is administered within 12 hours, preferably within 6 hours, more preferably within 3 hours, most preferably within 1 hour after the administration of the first drug,

Typically, the active ingredients are applied topically to the patient, i.e. to the affected areas of the skin.

Pharmaceutical compositions according to the invention may be suitable for oral, buccal, nasal, topical, ophthalmic or rectal administration. Preferably, the compositions are suitable for topical administration.

For oral administration, the pharmaceutical compositions of the present invention may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate. For ophthalmic administration the pharmaceutical compositions of the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration compounds may be formulated in an ointment such as petrolatum. For rectal administration the pharmaceutical compositions of the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

For topical administration the pharmaceutical compositions of the present invention may take the form of any formulation normally used for topical administration, in particular solutions, lotions, emulsions of liquid consistency, emulsions of semi-liquid consistency, emulsions of semi-solid consistency, emulsions of solid consistency, creams, gels or ointments. The emulsions are obtained by dispersion of an oil phase in water (O/W) or a water phase in oil (W/O). For example, some pharmaceutical compositions for topical administration contain an oil phase. Such pharmaceutical compositions may, for example, be water-in-oil emulsions (i.e. emulsions wherein the water is the dispersed phase and the oil in the dispersion medium) or be substantially non-aqueous.

Compositions for topical use in accordance with the invention may also contain one or more emollients, emulsifiers, thickeners and/or preservatives. The emollients are typically long chain alcohols, such as cetyl alcohol, stearyl alcohol and cetearyl alcohol; hydrocarbons such as petrolatum and light mineral oil; or acetylated lanolin. The total amount of emollient in the formulation is preferably about 5% to about 30%, and more preferably about 5% to about 10% by weight based on the total weight of the formulation. The emulsifier is typically a nonionic surface active agent, e.g., polysorbate 60 (available from Sigma Aldrich), sorbitan monostearate, polyglyceryl-4 oleate, and polyoxyethylene(4)lauryl ether or trivalent cationic. Generally the total amount of emulsifier is preferably about 2% to about 14%, and more preferably about 2% to about 6% by weight based on the total weight of the formulation. Pharmaceutically acceptable thickeners, such as Veegum.TM.K (available from R. T. Vanderbilt Company, Inc.), and long chain alcohols (i.e. cetyl alcohol, stearyl alcohol or cetearyl alcohol) can be used. The total amount of thickener present is preferably about 3% to about 12% by weight based on the total weight of the formulation. Preservatives such as methylparaben, propylparaben and benzyl alcohol can be present in the formulation.

Optionally, an additional solubilizing agent such as benzyl alcohol, lactic acid, acetic acid, stearic acid or hydrochloric acid can be included in the formulation. If an additional solubilizing agent is used, the amount present is preferably about 1% to about 12% by weight based on the total weight of the cream.

Optionally, the formulation can contain a humectant such as glycerin and skin penetration enhancers such as butyl stearate.

It is known to those skilled in the art that a single ingredient can perform more than one function in a composition, i.e., cetyl alcohol can serve both as an emollient and as a thickener.

The pharmaceutical composition of the invention optionally comprises an oil phase. In this case, typically the amount of oil in the composition is at least 10 wt. %, preferably at least 30 wt. %, more preferably at least 50 wt. %, more preferably at least 80 wt. %, based on the total weight of the composition. As used herein an oil phase is typically a liquid or solid phase which is substantially immiscible with water. More typically, an oil phase as used herein has a solubility in water at 25° C. of less than or equal to 1 mg/L, preferably less than 0.1 mg/L.

The oil phase in an emulsion may be any oil phase normally used in emulsions for topical administration. Such oil phases include, for example, hydrocarbon bases such as such as hard paraffin, soft paraffin, ceresine and microcrystalline wax, absorption bases such as lanolin and beeswax, emulsifying bases such as emulsifying wax and cetrimide, and vegetable oils such as olive oil, coconut oil, sesame oil, almond oil and peanut oil. Other oil phases useful in accordance with the invention are mineral oil, liquid petroleum, sorbitan monostearate, polysorbate 60, cetyl esters wax, cetearyl alcohol, benzyl alcohol and 2-octyldodecanol.

Those skilled in the art will understand that by varying the ratio of water to oil in an emulsion, the result could be deemed a lotion, a cream, or an ointment, by order of increasing proportion of oil. An emulsion comprising similar proportions of oil phase and water phase is usually deemed a cream, whereas an ointment will generally contain a substantially higher proportion of oil phase compared to water phase, for example greater than 60 wt. % oil phase, preferably greater than 70 wt. % oil phase, more preferably greater than 80 wt. % oil phase, based on the total weight of the oil phase and the water phase. A lotion will generally contain a lower proportion of oil phase than a cream, for example under 25 wt. % oil phase, under 20 wt. % oil phase, under 15 wt. % oil phase, under 10 wt. % oil phase or under 5 wt. % oil phase, based on the total weight of the oil phase and the water phase.

Generally, a cream for use according to the invention comprises an oil phase and a water phase mixed together to form an emulsion. Preferably, the amount of water present in a cream of the invention is about 45% to about 85% by weight based on the total weight of the cream, more preferably about 45 wt. % to about 65 wt. %, even more preferably about 45 wt. % to about 55 wt. %.

Where the composition is an ointment a pharmaceutically acceptable ointment base will be used. Examples of ointment bases include hydrocarbon bases such as such as hard paraffin, soft paraffin, ceresine and microcrystalline wax, absorption bases such as lanolin and beeswax, water-soluble bases such as polyethylene glycols (e.g. polyethylene glycol 200, 300, 400, 3350, 4000 or 6000), propylene glycol and polypropylene glycols, emulsifying bases such as emulsifying wax and cetrimide, and vegetable oils such as olive oil, coconut oil, sesame oil, almond oil and peanut oil. Mixtures of ointment bases can of course be used. The amount of ointment base present in an ointment of the invention is preferably about 60% to about 95% by weight based on the total weight of ointment, more preferably about 70 wt. % to about 90 wt. %, still more preferably about 75 wt. % to about 85 wt. %.

The pharmaceutical composition for use in accordance with the present invention may also be a lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

Specific exemplary, but non-limiting compositions include those in which the two active ingredients are incorporated into the following topical vehicles: (i) a vehicle comprising macrogol stearate 400, glycol. Monostearate 44-50, sorbitan monostearate and petroleum jelly (e.g., Unguentum Cordes®, which is an amphiphilic, hypoallergenic vehicle); and (ii) paraben-preserved water.

In the compositions and products according to the invention, the HMG-CoA reductase inhibitor and the cholesterol or pharmaceutically acceptable precursor thereof may each be present at a concentration of between 0.001 and 20% by weight, relative to the total weight of the composition or product, preferably between 0.01 and 10%, more preferably between 0.02 and 5% by weight, and more preferably still between 1 and 4% by weight. In a particular embodiment, each of the two active ingredients is present at a concentration of between 1 and 3% by weight (e.g. approximately 2% by weight of cholesterol and between 2% by weight of simvastatin or lovastatin).

Further Combination with a Vitamin D Agent

In a further aspect of the present invention, the combination therapy may further comprise administration of a vitamin D agent. Non-limiting examples of such compounds include vitamin D3 (cholecalciferol), calcitriol (1,25-dihydroxyvitamin D3), 1α,25-dihydroxycholecalciferol, alfacalcidol (1α-hydroxyvitamin D3), 24,25-dihydroxyvitamin D3, calcifediol, and vitamin D2.

Typically in a combination therapy involving a vitamin D agent, the combination therapy further involves both the HMG-CoA reductase inhibitor and cholesterol or pharmaceutically acceptable precursor thereof. However, it is also possible for the combination therapy to comprise administration of the vitamin D agent with: (a) the HMG-CoA reductase inhibitor; or (b) cholesterol or pharmaceutically acceptable precursor thereof.

When a vitamin D agent is to be co-administered with the HMG-CoA reductase inhibitor and/or cholesterol or pharmaceutically acceptable precursor thereof, then it can be administered either in combination with one or more of the other active ingredients, or separately to administration of any other active ingredient. There is no particular limitation on the timing of administration of the vitamin D agent. Preferably the vitamin D agent is to be administered topically.

Without being limited to theory, one possible explanation for the usefulness of vitamin D agents is as follows. It has previously been taught that topical vitamin D can be used to treat psoriasis in some cases (for example, Dovonex® cream). However, it has not previously been understood why such a treatment may work. In light of the present findings, suggesting the existence of imbalances in cholesterol metabolites in at least some patients, this may further result a lack of vitamin D in the skin due to related pathways for the production of these components. For instance, 7-dehydrocholesterol, a precursor of cholesterol (as discussed elsewhere herein) is also a precursor of vitamin D; genetic variants impacting on the content of 7-dehydrocholesterol may therefore modulate the content of either or both of cholesterol and vitamin D in the skin.

The present invention is explained in more detail in the following by referring to Examples, which are not to be construed as limitative.

### EXAMPLES

### Example 1

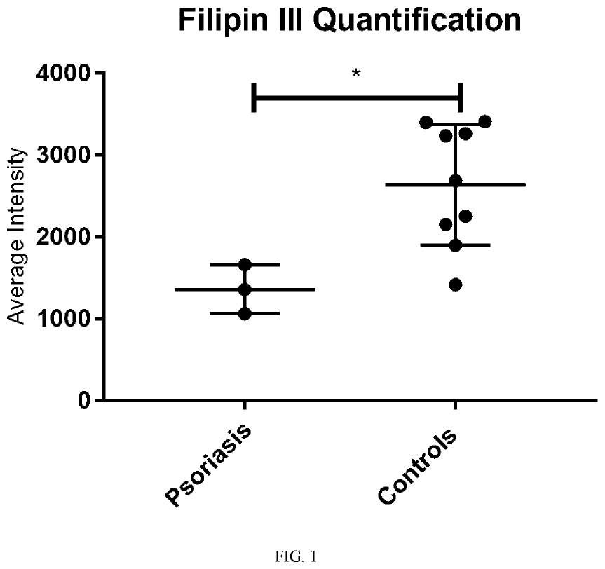

Experiments to compare the level of cholesterol in psoriasis patients against non-psoriasis controls were performed. Filipin III was used as a cholesterol stain as it is highly fluorescent and binds specifically to cholesterol. Cultured and immortalized primary keratinocytes from psoriasis patients as a group and individually were shown to have (statistically significantly) lower mean levels of cholesterol than cultured immortalized primary keratinocytes from grouped non-psoriasis controls, as shown in FIG. 1. This strongly suggests a final common functional pathway of cholesterol metabolite imbalance or dysregulation, and hence that psoriasis patients may be susceptible to treatment via therapies that contribute to optimizing the balance of cholesterol and its metabolites (and potentially also Vitamin D) of the skin.

### Example 2

Five patients with psoriasis were recruited for clinical study. Information about the patients, and treatment results, are summarized in Table 2 below.

Of the five patients, one was discovered to have developed psoriasis on top of a rare genetic skin condition. It therefore not being clear that he was a pure enough example, this patient was excluded from the study.

Methods

Five patients with psoriasis were recruited. Photographs were taken of all cases, in the four studied patients, as a baseline. Skin biopsy of affected skin was obtained for primary keratinocyte culture and immortalization with HPV to establish a stable cell line, as well as direct DNA and RNA extraction and histology. Information about the patients is summarised in Table 2 below.

Four of the five patients were treated with 2% cholesterol/2% simvastatin in Unguentum Merck, applied topically twice a day for a minimum period of three months in the first instance, and only applied to one designated area of affected skin, using the rest of the skin as an internal control. If there was no response in this time the treatment was stopped. Where there was a positive response the treatment has been continued, and extended gradually to different body areas. As noted above, the fifth patient was excluded and so not treated.

Results

As of December 2019, of the five patients, one had clear and somewhat prolonged improvement (patient 3 in Table 2), and a second had improvement in the form of less scaly plaques but no decrease in erythema or size (patient 1 in Table 2). One did not have a clear or prolonged response (patient 2 in Table 2). The remaining two are still in first three-month trial phase (patient 4 in Table 2) or about to start (patient 5 in Table 2).

As of December 2020, the following further updates were reported. Patient 3 in Table 2 again demonstrated a good response (after restarting treatment) and has continued treatment. No clear or prolonged response was found for patient 4 in Table 2. As noted above, patient 5 in Table 2 was excluded due to the presence of a potentially confounding rare genetic skin condition.

TABLE 2

| Patient | | | |
|---|---|---|---|
| No | Sex | Phenotype | Response to topical cholesterol/simvastatin |
| 1 | Female | Classical plaque | December 2019: Plaques less scaly, but no decrease |
| | | psoriasis | in erythema or size. |
| 2 | Male | Severe | December 2019: No response |
| | | palmoplantar | |
| | | psoriasis | |
| 3 | Male | Classical plaque | December 2019: Initial good response by report from |
| | | psoriasis | patient and parents, thereafter wore off. Going to |
| | | | restart with new pot of cholesterol/simvastatin. |
| | | | December 2020: Again good response and has |
| | | | continued treatment. |
| 4 | Female | Classic plaque | December 2019: Not yet completed the treatment |
| | | psoriasis | for 3 months, awaiting outcome |
| | | | December 2020: no response |
| 5 | Male | Patient excluded | — |
| | | from study | |

### Example 3

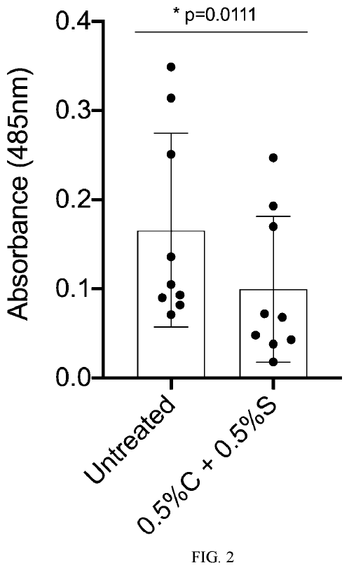

A WST-1 assay was conducted to study the proliferation rate of keratinocytes in culture. Psoriasis keratinocytes from three psoriasis patients were tested, both untreated and after 24 hours of incubation with 0.5% cholesterol in 100% ETOH and 0.5% simvastatin in DMSO (experiments were conducted in triplicate for each patient in both the untreated and treated samples). The results are shown in FIG. 2, demonstrating significantly reduced cellular proliferation as a result of treatment with the cholesterol/simvastatin combination.

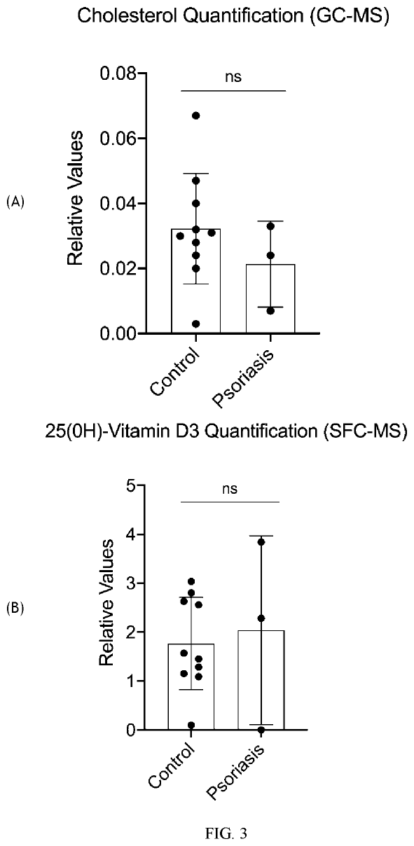

Total cholesterol and vitamin D3 concentrations of keratinocytes isolated from three psoriasis and ten control patients were measured respectively using Gas Chromatography Mass Spectrometry (GC-MS) and Supercritical Fluid Chromatography Mass Spectrometry (SFC-MS) normalised to live cell count. The results are shown in FIG. 3. Although statistical significance was not established, possibly due to sample size, the results showed some trend towards reduced cholesterol in psoriasis patients compared to controls.

1-13. (canceled)

14. A method of treating a patient suffering from psoriasis which method comprises co-administering to said patient (a) an HMG-CoA reductase inhibitor, and (b) cholesterol or a pharmaceutically acceptable precursor thereof.

15. (canceled)

16. A method according to claim 14, wherein the method comprises simultaneous, concurrent, separate or sequential administration of a product comprising (a) the HMG-CoA reductase inhibitor, and (b) the cholesterol or a pharmaceutically acceptable precursor thereof, as a combined preparation.

17-20. (canceled)

21. A method according to claim 14, wherein the HMG-CoA reductase inhibitor is simvastatin, lovastatin, atorvastatin, cerivastatin, fluvastatin, mevastatin, pitavastatin, pravastatin or rosuvastatin, or a pharmaceutically acceptable salt or ester thereof.

22. A method according to claim 14, wherein the HMG-CoA reductase inhibitor is simvastatin, lovastatin, atorvastatin, fluvastatin, pravastatin or rosuvastatin, or a pharmaceutically acceptable salt or ester thereof.

23. A method according to claim 14, wherein the HMG-CoA reductase inhibitor is simvastatin or lovastatin, or a pharmaceutically acceptable salt or ester thereof.

24. A method according to claim 14, wherein the cholesterol or a pharmaceutically acceptable precursor thereof is cholesterol, a prodrug of cholesterol or an intermediate in the in vivo production of cholesterol from mevalonate.

25. A method according to claim 14, wherein the cholesterol or a pharmaceutically acceptable precursor thereof is cholesterol.

26. A method according to claim 14, wherein the HMG-CoA reductase inhibitor is simvastatin or lovastatin and the cholesterol or a pharmaceutically acceptable precursor thereof is cholesterol.

27. A method according to claim 14, wherein the method comprises topically administering the HMG-CoA reductase inhibitor and the cholesterol or a pharmaceutically acceptable precursor thereof.

28. A method according to claim 14, wherein said patient has abnormal cholesterol metabolism.

29. A method according to claim 14, wherein said patient has a variant in one or more genes associated with cholesterol metabolism

30. A method according to claim 14, wherein said patient has a variant in one or more genes selected from the group consisting of ABCG8, ACADL, APOF, CAD, CARD14, CBR3, CDSN, CLN8, CSTA, CYB5R2, CYP7B1, FBXW7, FDFT1, FDPS, FDX1L, HMGCR, KRT2, KRT6C, LDLR, LEP, LPL, LRP5, LRP8, OSBP, OSBP2, PKP1, PROM2, PTCH1, RORC, SCARB1, SERPINA12, SORL1, TRERF1, UMPS, TGFB1, PMVK, MVK and NSDHL.

31. A method according to claim 14, wherein said patient has a variant in one or more genes selected from the group consisting of ABCG8, ACADL, APOF, CAD, CARD14, CBR3, CDSN, CLN8, CSTA, CYB5R2, CYP7B1, FBXW7, FDFT1, FDPS, FDX1L, HMGCR, KRT2, KRT6C, LDLR, LEP, LPL, LRP5, LRP8, OSBP, OSBP2, PKP1, PROM2, PTCH1, RORC, SCARB1, SERPINA12, SORL1, TRERF1 and UMPS.
